(19) United States
(12) Patent Application Publication
(10) Pub. No.: US 2003/0054551 A1
(43) Pub. Date: Mar. 20, 2003

(54) Effect of growth hormone and IGF-1 on neural stem cells

(75) Inventors: Tetsuro Shingo, Aoe (JP); Samuel Weiss, Calgary (CA)

Correspondence Address: BURNS DOANE SWECKER & MATHIS L L P, POST OFFICE BOX 1404, ALEXANDRIA, VA, 22313-1404, US

(73) Assignee: Stem Cell Therapeutics Inc., Calgary (CA)

(21) Appl. No.: 10/231,492

(22) Filed: Aug. 30, 2002

Related U.S. Application Data
(60) Provisional application No. 60/323,503, filed on Sep. 18, 2001.
(60) Provisional application No. 60/386,404, filed on Jun. 7, 2002.

(51) Int. Cl. A61K 38/18; C12N 5/08; A61K 38/27; A61K 38/30; A61K 31/56; A61K 31/7076; A61K 31/405

(52) U.S. Cl. 435/368; 514/12; 514/182; 514/419; 514/47

(57) ABSTRACT

The present invention provides a method of increasing neural stem cell numbers by using growth hormone and/or IGF-1. The method can be practiced in vivo to obtain more neural stem cells in situ, which can in turn produce more neurons or glial cells to compensate for lost or dysfunctional neural cells. The method can also be practiced in vitro to produce a large number of neural stem cells in culture. The cultured stem cells can be used, for example, for transplantation treatment of patients or animals suffering from neurodegenerative diseases or conditions.

### RELATED APPLICATIONS

This application claims the benefit of U.S. Provisional Applications Serial No. 60/323,503, filed Sep. 18, 2001, and Serial No. 60/386,404, filed Jun. 7, 2002. The entire disclosure of each of these priority applications is hereby incorporated by reference.

### FIELD OF THE INVENTION

The present invention relates to methods of increasing neural stem cell numbers by using growth hormone (GH) and/or insulin-like growth factor 1 (IGF-1), as well as methods for treating or ameliorating neurodegenerative diseases or conditions.

### REFERENCES

U.S. Patent Application Publication No. 2002 0098178 A1.

U.S. Pat. No. 5,023,252.

U.S. Pat. No. 5,128,242.

U.S. Pat. No. 5,198,542.

U.S. Pat. No. 5,208,320.

U.S. Pat. No. 5,231,178.

U.S. Pat. No. 5,268,164.

U.S. Pat. No. 5,326,860.

U.S. Pat. No. 5,473,054.

U.S. Pat. No. 5,506,107.

U.S. Pat. No. 5,506,206.

U.S. Pat. No. 5,527,527.

U.S. Pat. No. 5,547,935.

U.S. Pat. No. 5,614,184.

U.S. Pat. No. 5,623,050.

U.S. Pat. No. 5,686,416.

U.S. Pat. No. 5,723,115.

U.S. Pat. No. 5,773,569.

U.S. Pat. No. 5,750,376.

U.S. Pat. No. 5,801,147.

U.S. Pat. No. 5,833,988.

U.S. Pat. No. 5,837,460.

U.S. Pat. No. 5,851,832.

U.S. Pat. No. 5,885,574.

U.S. Pat. No. 5,955,346.

U.S. Pat. No. 5,977,307.

U.S. Pat. No. 5,980,885.

U.S. Pat. No. 6,015,555.

U.S. Pat. No. 6,048,971.

U.S. Pat. No. 6,191,106.

U.S. Pat. No. 6,242,563.

U.S. Pat. No. 6,329,508.

U.S. Pat. No. 6,333,031.

U.S. Pat. No. 6,413,952.

WO 90/05185.

WO 96 40231.

WO 97 48729.

Bernichtein, S., et al. S179D-human PRL, a pseudophosphorylated human PRL analog, is an agonist and not an antagonist. Endocrinology 142(9):3950-3963 (2001).

Cunningham, B. C., et al. High-resolution epitope mapping of hGH-receptor interactions by alanine-scanning mutagenesis. Science 244(4908):1081-5 (1989a).

Cunningham, B. C., et al. Receptor and antibody epitopes in human growth hormone identified by homolog-scanning mutagenesis. Science 243(4896):1330-1336 (1989b).

Fernandez-Pol, J. A. Epidermal growth factor receptor of A431 cells. Characterization of a monoclonal anti-receptor antibody noncompetitive agonist of epidermal growth factor action. J. Biol. Chem. 260(8):5003-5011 (1985).

Gray, G. L., et al. Periplasmic production of correctly processed human growth hormone in Escherichia coli: natural and bacterial signal sequences are interchangeable. Gene 39(2-3):247-254 (1985).

Goeddel, D. V., et al. Direct expression in Escherichia coli of a DNA sequence coding for human growth hormone. Nature 281(5732):544-548 (1979).

Johnson, D. L., et al. Erythropoietin mimetic peptides and the future. Nephrol. Dial. Transplant. 15(9): 1274-1277 (2000).

Kaushansky, K. Hematopoietic growth factor mimetics. Ann. N. Y. Acad. Sci. 938:131-138 (2001).

Kolb, B., et al. Nerve growth factor treatment prevents dendritic atrophy and promotes recovery of function after cortical injury. Neuroscience 76(4):1139-1151 (1997).

Lim, D. A., et al., “Noggin antagonizes BMP signaling to create a niche for adult neurogenesis”, Neuron 28: 713-726 (2000).

Livnah, O., et al. Functional mimicry of a protein hormone by a peptide agonist: the EPO receptor complex at 2.8 A. Science 273(5274):464-471 (1996).

Mode, A., et al. The human growth hormone (hGH) antagonist G120RhGH does not antagonize GH in the rat, but has paradoxical agonist activity, probably via the prolactin receptor. Endocrinology 137(2):447-454 (1996).

Moro, O., et al. Maxadilan, the vasodilator from sand flies, is a specific pituitary adenylate cyclase activating peptide type I receptor agonist. J. Biol. Chem. 272(2):966-70 (1997).

Nyberg, F., “Aging effects on growth hormone receptor binding in the brain”, Exp. Gerontol 32: 521-528 (1997).

Nyberg, F., “Growth hormone in the brain: characteristics of specific brain targets for the hormone and their functional significance”, Front Neuroendocrinol. 21: 330-348 (2000).

Shingo, T., et al. Erythropoietin regulates the in vitro and in vivo production of neuronal progenitors by mammalian forebrain neural stem cells. J. Neurosci. 21(24):9733-9743 (2001).

Tropepe, V., et al., “Transforming growth factor-alpha null and senescent mice show decreased neural progenitor cell proliferation in the forebrain subependyma”, J. Neurosci. 17: 7850-7859 (1997).

Wrighton, N. C., et al. Small peptides as potent mimetics of the protein hormone erythropoietin. Science 273(5274):458-464 (1996).

All of the publications, patents and patent applications cited above or elsewhere in this application are herein incorporated by reference in their entirety to the same extent as if the disclosure of each individual publication, patent application or patent was specifically and individually indicated to be incorporated by reference in its entirety.

### BACKGROUND OF THE INVENTION

In recent years, neurodegenerative disease has become an important concern due to the expanding elderly population which is at greatest risk for these disorders. Neurodegenerative diseases include the diseases which have been linked to the degeneration of neural cells in particular locations of the central nervous system (CNS), leading to the inability of these cells to carry out their intended function. These diseases include Alzheimer's Disease, Multiple Sclerosis (MS), Huntington's Disease, Amyotrophic Lateral Sclerosis, and Parkinson's Disease. In addition, probably the largest area of CNS dysfunction (with respect to the number of affected people) is not characterized by a loss of neural cells but rather by abnormal functioning of existing neural cells. This may be due to inappropriate firing of neurons, or the abnormal synthesis, release, and processing of neurotransmitters. These dysfunctions may be the result of well studied and characterized disorders such as depression and epilepsy, or less understood disorders such as neurosis and psychosis. Moreover, brain injuries often result in the loss of neural cells, the inappropriate functioning of the affected brain region, and subsequent behavior abnormalities.

Consequently, it is desirable to supply neural cells to the brain to compensate for degenerate or lost neurons in order to treat neurodegenerative diseases or conditions. One approach to this end is to transplant neural cells into the brain of the patient. This approach requires a source of large amounts of neural cells, preferably from the same individual or a closely related individual such that host-versus-graft or graft-versus-host rejections can be minimized. As it is not practical to remove a large amount of neurons or glial cells from one person to transplant to another, a method to culture large quantity of neural cells is necessary for the success of this approach.

Another approach is to induce the production of neural cells in situ to compensate for the lost or degenerate cells. This approach requires extensive knowledge about whether it is possible to produce neural cells in brains, particularly adult brains, and how.

The development of techniques for the isolation and in vitro culture of multipotent neural stem cells (for example, see U.S. Pat. Nos. 5,750,376; 5,980,885; 5,851,832) significantly increased the outlook for both approaches. It was discovered that fetal brains can be used to isolate and culture multipotent neural stem cells in vitro. Moreover, in contrast to the long time belief that adult brain cells are not capable of replicating or regenerating brain cells, it was found that neural stem cells may also be isolated from brains of adult mammals. These stem cells, either from fetal or adult brains, are capable of self-replicating. The progeny cells can again proliferate or differentiate into any cell in the neural cell lineage, including neurons, astrocytes and oligodendrocytes. Therefore, these findings not only provide a source of neural cells which can be used in transplantations, but also demonstrate the presence of multipotent neural stem cells in adult brain and the possibility of producing neurons or glial cells from these stem cells in situ.

It is therefore desirable to develop methods of efficiently producing neural stem cells for two purposes: to obtain more stem cells and hence neural cells which can be used in transplantation therapies, and to identify methods which can be used to produce more stem cells in situ.

### SUMMARY OF THE INVENTION

The present invention provides a method of increasing neural stem cell numbers by using growth hormone and/or IGF-1. The method can be practiced in vivo to obtain more neural stem cells in situ, which can in turn produce more neurons or glial cells to compensate for lost or dysfunctional neural cells. The method can also be practiced in vitro to produce a large number of neural stem cells in culture. The cultured stem cells can be used, for example, for transplantation treatment of patients or animals suffering from neurodegenerative diseases or conditions.

Accordingly, one aspect of the present invention provides a method of increasing neural stem cell number, comprising providing an effective amount of a growth hormone and/or IGF-1 to at least one neural stem cell under conditions which result in an increase in the number of neural stem cells. The neural stem cell may be located in the brain of a mammal, in particular in the subventricular zone of the brain of the mammal. Preferably, the growth hormone and/or IGF-1 is administered to the ventricle of the brain. Although mammals of all ages can be subjected to this method, it is preferable that the mammal is not an embryo. More preferably, the mammal is an adult.

The mammal may suffer from or be suspected of having a neurodegenerative disease or condition. The disease or condition may be a brain injury, such as stroke or an injury caused by a brain surgery. The disease or condition may be aging, which is associated with a significant reduction in the number of neural stem cells. The disease or condition can also be a neurodegenerative disease, particularly Alzheimer's disease, multiple sclerosis, Huntington's disease, amyotrophic lateral sclerosis, or Parkinson's disease.

Alternatively, the neural stem cell may be in a culture in vitro.

Whether the method is used in vivo or in vitro, other factors may be applied in combination with the growth hormone/IGF-1, such as erythropoietin, cyclic AMP, pituitary adenylate cyclase activating polypeptide (PACAP), serotonin, bone morphogenetic protein (BMP), epidermal growth factor (EGF), transforming growth factor alpha (TGFα), fibroblast growth factor (FGF), estrogen, prolactin, and/or ciliary neurotrophic factor (CNTF). The additional factor is preferably selected from the group consisting of EGF, erythropoietin, prolactin and PACAP. More preferably, the additional factor is EGF or prolactin.

The growth hormone, IGF-1, and/or the additional factor can be provided by any method established in the art. For example, they can be administered intravascularly, intrathecally, intravenously, intramuscularly, subcutaneously, intraperitoneally, topically, orally, rectally, vaginally, nasally, by inhalation or into the brain. The administration is preferably performed systemically, particularly by subcutaneous administration. The factor can also be provided by administering to the mammal an effective amount of an agent that can increase the amount of the endogenous factor in the mammal. For example, the level of prolactin in an animal can be increased by using prolactin releasing peptide.

When the factor is not directly delivered into the brain, a blood brain barrier permeabilizer can be optionally included to facilitate entry into the brain. Blood brain barrier permeabilizers are known in the art and include, by way of example, bradykinin and the bradykinin agonists described in U.S. Pat. Nos. 5,686,416; 5,506,206 and 5,268,164 (such as NH₂-arginine-proline-hydroxyproxyproline-glycine-thienylalanine-serine-proline-4-Me-tyrosineψ(CH₂NH)-arginine-COOH). Alternatively, the factors can be conjugated to the transferrin receptor antibodies as described in U.S. Pat. Nos. 6,329,508; 6,015,555; 5,833,988 or 5,527,527. The factors can also be delivered as a fusion protein comprising the factor and a ligand that is reactive with a brain capillary endothelial cell receptor, such as the transferrin receptor (see, e.g., U.S. Pat. No. 5,977,307).

Another aspect of the present invention provides a method of treating or ameliorating a neurodegenerative disease or condition in a mammal, comprising administering an effective amount of a growth hormone and/or IGF-1 to the brain of the mammal. The disease or condition may be a brain injury, such as stroke or an injury caused by a brain surgery. The disease or condition may be aging, which is associated with a significant reduction in the number of neural stem cells. The disease or condition can also be a neurodegenerative disease, particularly Alzheimer's Disease, Multiple Sclerosis, Huntington's Disease, Amyotrophic Lateral Sclerosis, or Parkinson's Disease. Preferably, the neurodegenerative condition is aging or stroke.

### BRIEF DESCRIPTION OF THE DRAWINGS

FIG. 1

(A) Time course for neural stem cell decline in male and female C57BL/6J mice.

(B) Three other mouse strains show a similar pattern of neural stem cell decline.

(C) Neural stem cells from aging animals are multipotent but show reduced expansion/self renewal.

### DETAILED DESCRIPTION OF THE INVENTION

The present invention provides a method of increasing neural stem cell numbers by using growth hormone or insulin-like growth factor 1 (IGF-1). The method can be practiced in vivo to obtain more neural stem cells in situ, which can in turn produce more neurons or glial cells to compensate for lost or dysfunctional neural cells. The method can also be practiced in vitro to produce a large number of neural stem cells in culture. The cultured stem cells can be used, for example, for transplantation treatment of patients or animals suffering from neurodegenerative diseases or conditions.

Prior to describing the invention in further detail, the terms used in this application are defined as follows unless otherwise indicated.

Definitions

A “neural stem cell” is a stem cell in the neural cell lineage. A stem cell is a cell which is capable of reproducing itself. In other words, daughter cells which result from stem cell divisions include stem cells. The neural stem cells are capable of ultimately differentiating into all the cell types in the neural cell lineage, including neurons, astrocytes and oligodendrocytes (astrocytes and oligodendrocytes are collectively called glia or glial cells). Thus, the neural stem cells referred to herein are multipotent neural stem cells.

A “neurosphere” is a group of cells derived from a single neural stem cell as the result of clonal expansion. A “primary neurosphere” refers to the neurospheres generated by plating as primary cultures brain tissue which contains neural stem cells. The method for culturing neural stem cells to form neurospheres has been described in, for example, U.S. Pat. No. 5,750,376. A “secondary neurosphere” refers to the neurospheres generated by dissociating primary neurospheres and allowing the individual dissociated cells to form neurospheres again.

A polypeptide which shares “substantial sequence similarity” with a native factor is at least about 30% identical with the native factor at the amino acid level. The polypeptide is preferably at least about 40%, more preferably at least about 60%, yet more preferably at least about 70%, and most preferably at least about 80% identical with the native factor at the amino acid level.

The phrase “percent identity” or “% identity” of an analog or variant with a native factor refers to the percentage of amino acid sequence in the native factor which are also found in the analog or variant when the two sequences are aligned. Percent identity can be determined by any methods or algorithms established in the art, such as LALIGN or BLAST.

A polypeptide possesses a “biological activity” of a native factor if it is capable of exerting any of the biological activities of the native factor, or being recognized by a polyclonal antibody raised against the native factor. Preferably, the polypeptide is capable of specifically binding to the receptor for the native factor in a receptor binding assay.

A “growth hormone” is a polypeptide which (1) shares substantial sequence similarity with a native mammalian growth hormone, particularly the native human growth hormone; and (2) possesses a biological activity of the native mammalian growth hormone. The native human growth hormone is a polypeptide containing 191 amino acids in a single chain and a molecular weight of about 22 kD (Goeddel et al., 1979; Gray et al., 1985). Thus, the term “growth hormone” encompasses growth hormone analogs which are the deletional, insertional, or substitutional mutants of the native growth hormone. Furthermore, the term “growth hormone” encompasses the growth hormones from other species and the naturally occurring variants thereof.

An “IGF-1” is a polypeptide which (1) shares substantial sequence similarity with a native mammalian IGF-1, particularly the native human IGF-1; and (2) possesses a biological activity of the native mammalian IGF-1. The native human IGF-1 is a polypeptide of 70 amino acids with a molecular weight of 7648 daltons (see, for example, U.S. Pat. No. 5,231,178). A polypeptide which shares “substantial sequence similarity” with the native human IGF-1 is at least about 30% identical with a native mammalian IGF-1 at the amino acid level. The IGF-1 is preferably at least about 40%, more preferably at least about 60%, yet more preferably at least about 70%, and most preferably at least about 80% identical with the native mammalian IGF-1 at the amino acid level. Thus, the term “IGF-1” encompasses IGF-1 analogs which are the deletional, insertional, or substitutional mutants of the native IGF-1. Furthermore, the term “IGF-1” encompasses the IGF-1s from other species and the naturally occurring variants thereof.

An “EGF” means a native EGF or any EGF analog or variant that shares a substantial amino acid sequence similarity with a native EGF, as well as at least one biological activity with the native EGF, such as binding to the EGF receptor. Particularly included as an EGF is the native EGF of any species, TGFα, or recombinant modified EGF. Specific examples include, but are not limited to, the recombinant modified EGF having a deletion of the two C-terminal amino acids and a neutral amino acid substitution at position 51 (particularly EGF51gln51; U.S. Patent Application Publication No. 20020098178A1), the EGF mutein (EGF-X₁₆) in which the His residue at position 16 is replaced with a neutral or acidic amino acid (U.S. Pat. No. 6,191,106), the 52-amino acid deletion mutant of EGF which lacks the amino terminal residue of the native EGF (EGF-D), the EGF deletion mutant in which the N-terminal residue as well as the two C-terminal residues (Arg-Leu) are deleted (EGF-B), the EGF-D in which the Met residue at position 21 is oxidized (EGF-C), the EGF-B in which the Met residue at position 21 is oxidized (EGF-A), heparin-binding EGF-like growth factor (HB-EGF), betacellulin, amphiregulin, neuregulin, or a fusion protein comprising any of the above. Other useful EGF analogs or variants are described in U.S. Patent Application Publication No. 20020098178A1, and U.S. Pat. Nos. 6,191,106 and 5,547,935.

In addition, an “EGF” may also be a functional agonist of a native mammalian EGF receptor. For example, the functional agonist may be an activating amino acid sequence disclosed in U.S. Pat. No. 6,333,031 for the EGF receptor, or an antibody that has agonist activities for the EGF receptor (Fernandez-Pol, 1985 and U.S. Pat. No. 5,723,115).

A “PACAP” means a native PACAP or any PACAP analog or variant that shares a substantial amino acid sequence similarity with a native PACAP, as well as at least one biological activity with the native PACAP, such as binding to the PACAP receptor. Useful PACAP analogs and variants include, without being limited to, the 38 amino acid and the 27 amino acid variants of PACAP (PACAP38 and PACAP27, respectively), and the analogs and variants disclosed in, e.g., U.S. Pat. Nos. 5,128,242; 5,198,542; 5,208,320; 5,326,860; 5,623,050; 5,801,147 and 6,242,563.

In addition, a “PACAP” may also be a functional agonist of a native mammalian PACAP receptor. For example, the functional agonist may be maxadilan, a polypeptide that acts as a specific agonist of the PACAP type-1 receptor (Moro et al., 1997).

An “erythropoietin (EPO)” means a native EPO or any EPO analog or variant that shares a substantial amino acid sequence similarity with a native EPO, as well as at least one biological activity with the native EPO, such as binding to the EPO receptor. Erythropoietin analogs and variants are disclosed, for example, in U.S. Pat. Nos. 6,048,971 and 5,614,184.

In addition, an “EPO” may also be a functional agonist of a native mammalian EPO receptor. For example, the functional agonist may be EMP1 (EPO mimetic peptide 1, Johnson et al., 2000); one of the short peptide mimetics of EPO as described in Wrighton et al., 1996 and U.S. Pat. No. 5,773,569; any small molecular EPO mimetic as disclosed in Kaushansky, 2001; an antibody that activates the EPO receptor as described in U.S. Pat. No. 5,885,574, WO 96/40231, WO 97/48729, Fernandez-Pol, 1985 or U.S. Pat. No. 5,723,115; an activating amino acid sequence as disclosed in U.S. Pat. No. 6,333,031 for the EPO receptor; a metal complexed receptor ligand with agonist activities for the EPO receptor (U.S. Pat. No. 6,413,952), or a ligand for the EPO receptor as described in U.S. Pat. Nos. 5,506,107 and 5,837,460.

A “prolactin” is a polypeptide which (1) shares substantial sequence similarity with a native mammalian prolactin, preferably the native human prolactin; and (2) possesses a biological activity of the native mammalian prolactin. The native human prolactin is a 199-amino acid polypeptide synthesized mainly in the pituitary gland. Thus, the term “prolactin” encompasses prolactin analogs which are the deletional, insertional, or substitutional mutants of the native prolactin. Furthermore, the term “prolactin” encompasses the prolactins from other species and the naturally occurring variants thereof.

In addition, a “prolactin” may also be a functional agonist of a native mammalian prolactin receptor. For example, the functional agonist may be an activating amino acid sequence disclosed in U.S. Pat. No. 6,333,031 for the prolactin receptor; a metal complexed receptor ligand with agonist activities for the prolactin receptor (U.S. Pat. No. 6,413,952); G120RhGH, which is an analog of human growth hormone but acts as a prolactin agonist (Mode et al., 1996); or a ligand for the prolactin receptor as described in U.S. Pat. Nos. 5,506,107 and 5,837,460.

“Enhancing” the formation of a cell type means increasing the number of the cell type. Thus, a factor can be used to enhance neuron formation if the number of neurons in the presence of the factor is larger than the number of neurons absent the factor. The number of neurons in the absence of the factor may be zero or more.

A “neurodegenerative disease or condition” is a disease or medical condition associated with neuron loss or dysfunction. Examples of neurodegenerative diseases or conditions include neurodegenerative diseases, brain injuries or CNS dysfunctions. Neurodegenerative diseases include, for example, Alzheimer's disease, multiple sclerosis (MS), macular degeneration, glaucoma, diabetic retinopathy, peripheral neuropathy, Huntington's disease, amyotrophic lateral sclerosis, and Parkinson's disease. Brain injuries include, for example, stroke (e.g., hemorrhagic stroke, focal ischemic stroke or global ischemic stroke) and traumatic brain injuries (e.g. injuries caused by a brain surgery or physical accidents). CNS dysfunctions include, for example, depression, epilepsy, neurosis and psychosis.

“Treating or ameliorating” means the reduction or complete removal of the symptoms of a disease or medical condition.

A mammal “suspected of having a neurodegenerative disease or condition” is a mammal which is not officially diagnosed of the neurodegenerative disease or condition but shows a symptom of the neurodegenerative disease or condition, is susceptible to the neurodegenerative disease or condition due to family history or genetic predisposition, or has had the neurodegenerative disease or condition before and is subject to the risk of recurrence.

“Transplanting” a composition into a mammal refers to introducing the composition into the body of the mammal by any method established in the art. The composition being introduced is the “transplant”, and the mammal is the “recipient”. The transplant and the recipient may be syngeneic, allogeneic or xenogeneic. Preferably, the transplantation is an autologous transplantation.

An “effective amount” is an amount of a therapeutic agent sufficient to achieve the intended purpose. For example, an effective amount of a growth hormone to increase the number of neural stem cells is an amount sufficient, in vivo or in vitro, as the case may be, to result in an increase in neural stem cell number. An effective amount of a growth hormone to treat or ameliorate a neurodegenerative disease or condition is an amount of the growth hormone sufficient to reduce or remove the symptoms of the neurodegenerative disease or condition. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

Methods

The aging brain undergoes numerous changes that lead to reduced function and enhanced susceptibility to acute injury and neurodegenerative disease. For example, as is the case for many sensory systems, aging results in diminished olfactory performance. Furthermore, olfactory dysfunction is a hallmark of forebrain neurodegenerative disease, such as Alzheimer's, Parkinson's and Huntington's diseases. The periglomerular interneurons of the olfactory bulb, like the granule cells of the hippocampal dentate gyrus, have been known to turn over and be replenished throughout life in the adult mammal. The source of the periglomerular interneurons are neural stem cells in the subventricular zone, which undergo neurogenesis to form new neural cells and migrate along the rostral migratory stream to the olfactory bulb. Therefore, olfactory dysfunction in mammals at high age or neurodegenerative diseases may be linked to reduced number of neural stem cells in the subventricular zone.

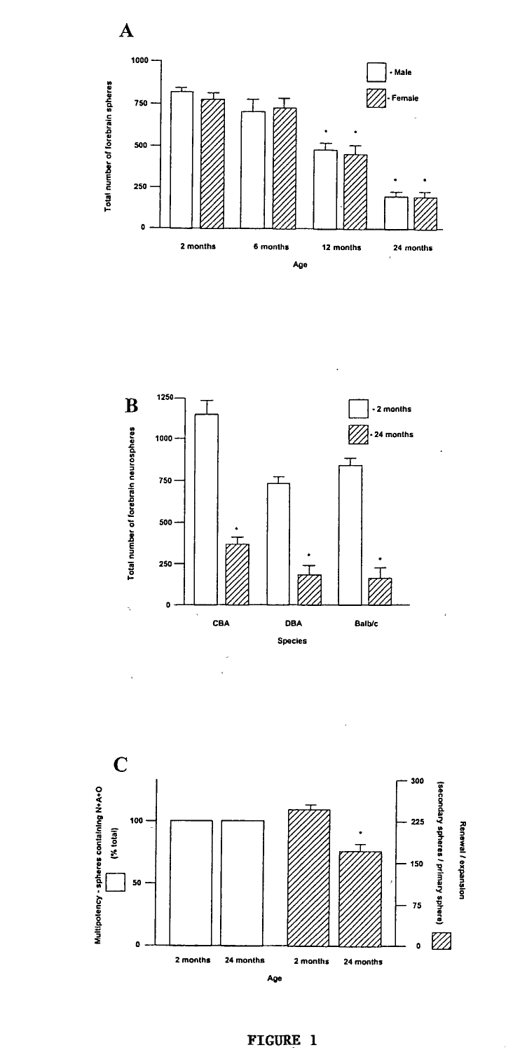

We therefore investigated the level of neural stem cells in mice at various ages (Example 1). As shown in FIG. 1A, aged mice have significantly less neural stem cells than their young adult counterparts, and the levels of neural stem cells are comparable between the male and female mice at each age. This finding was confirmed using three different strains of mice (FIG. 1B), indicating that the age-related reduction in stem cell number is not a strain-specific phenomenon.

This result is contrary to the report of Tropepe and colleagues (Tropepe et al., 1997), who compared the SVZs of senescent mice (23-25 month) and young adults (2-4 months). They reported that proliferation in the SVZ and the resultant new neurons in the olfactory bulb were substantially reduced in old mice, but the number of EGF-generated neurospheres derived from the SVZ was unchanged.

We also examined whether the neural stem cells harvested from different ages have the same biological activities. The neural stem cells from aged mice are still capable of differentiating into all three major kinds of mature neural cells, neurons, astrocytes and oligodendrocytes (FIG. 1C), but the ability to self-renew is reduced.

The number of neural stem cells can be increased by using growth hormone. Growth hormone receptors are expressed in the adult choroid plexus and the subventricular zone, and receptor expression decreases with aging (Nyberg, 1997; Nyberg, 2000). By infusing growth hormone into the ventricles in the presence of BrdU and subsequently determining the number of BrdU positive cells, we found that growth hormone was capable of inducing proliferation in the subventricular zone. The number of proliferating cells also increased in the rostral migratory stream, suggesting that growth hormone induced not only proliferation of neural stem cells but also migration of the progeny cells. As migration of the progeny of neural stem cells along the rostral migratory stream is part of the neurogenesis process in the adult mammalian brain, these results indicate that growth hormone resulted in elevated level of neural stem cell as well as neurogenesis.

Accordingly, the present invention provides a method of increasing neural stem cell numbers. This method can be used to increase neural stem cell number in vivo to result in a larger pool of neural stem cells in the brain. This larger pool of neural stem cells can subsequently generate more neural cells, either neurons or glial cells, than would a population of stem cells without growth hormone. The neural cells, in turn, can compensate for lost or degenerate neural cells which are associated with neurodegenerative diseases and conditions, including nervous system injuries.

Growth hormone can also be used to increase neural stem cell numbers in vitro. The resulting stem cells can be used to produce more neurons and/or glial cells in vitro, or used in transplantation procedures into humans or animals suffering from neurodegenerative diseases or conditions. It is preferable that neural stem cells produced according to the present invention, rather than neurons or glial cells, are transplanted. Once neural stem cells are transplanted, growth and/or differentiation factors can be administered in vivo to further increase the number of stem cells, or to selectively enhance neuron formation or glial cell formation. For example, we have found that erythropoietin induces selective production of neurons over glial cells. Cyclic AMP and factors which enhance the cAMP pathway, such as pituitary adenylate cyclase activating polypeptide (PACAP) and serotonin, are also good candidates for selectively promoting neuron production. On the other hand, bone morphogenetic protein (BMP) has been reported to inhibit neuron production and enhance glial production by adult subventricular zone cells (Lim et al., 2000).

Accordingly, the present invention also provides a method for treating or ameliorating a neurodegenerative disease or condition in a mammal. This can be achieved, for example, by administering an effective amount of a growth hormone to the brain of the mammal, or transplanting neural stem cells, neurons and/or glial cells produced according to the present invention to the mammal. Preferably, neural stem cells are transplanted.

One particularly interesting neurodegenerative condition is aging. Since the number of neural stem cells in the subventricular zone is significantly reduced in aged mice, it will be of particular interest to ameliorate problems associated with aging by increasing neural stem cell numbers with growth hormone.

Another particularly important application of the present invention is the treatment and/or amelioration of brain injuries, such as stroke. As shown in Example 5, growth hormone, or the combination of growth hormone and EPO, increased neurogenesis in the brain of animals that suffered from a chemically induced stroke. Furthermore, these animals also showed significant improvement in a motor-related symptom, demonstrating the effect of the present invention in treatment of brain injuries.

Growth hormone is a major regulator of IGF-1 secretion in the brain. We found that neural stem cells robustly express both growth hormone receptors and IGF-1 receptors (Example 4), indicating that these cells respond to both hormones. Without being limited to a theory, the effect of growth hormone on neural stem cells as described above may be mediated, completely or partially, through IGF-1. Accordingly, the present invention also provides methods of increasing neural stem cell number by using IGF-1, and methods of treating or ameliorating neurodegenerative diseases or conditions by using IGF-1.

Also encompassed in the present invention are methods to increase neural stem cell numbers or treating/ameliorating neurodegenerative diseases or conditions by using chemical compounds or other factors which are known to increase the level of growth hormones or IGF-1 in mammals. Preferably, these compounds or factors are capable of increasing growth hormone or IGF-1 concentrations in the brain.

Compositions

The present invention provides compositions that comprises growth hormone and/or IGF-1, and at least one additional factor. The additional factor is capable of increasing neural stem cell number or enhancing neural stem cell differentiation to neurons or glial cells. The additional factor is preferably erythropoietin, EGF, PACAP, and/or prolactin.

Growth hormone is a polypeptide hormone in the growth hormone/prolactin family. The growth hormone useful in the present invention includes any growth hormone analog or variant which is capable of increasing neural stem cell number. A growth hormone analog or variant is a polypeptide which contains at least about 30% of the amino acid sequence of a native mammalian growth hormone, and which possesses a biological activity of the native mammalian growth hormone. Preferably, the biological activity of growth hormone is the ability to bind growth hormone receptors. Specifically included as growth hormones are the naturally occurring growth hormone variants and growth hormones from various species, including but not limited to, human, other primates, rat, mouse, sheep, pig, and cattle. Human GH variants and analogs are well known in the art (for example, see Cunningham et al., 1989a; Cunningham et al., 1989b; WO 90/05185; and U.S. Pat. No. 5,506,107).

The IGF- 1 useful in the present invention may be the native IGF-1, or any analog or variant of the native IGF-1 which has at least 30% of the amino acid sequence of a native mammalian IGF-1 as well as a biological activity of the native mammalian IGF-1. IGF-1 analogs and variants are well known in the art (see, for example, U.S. Pat. No. 5,473,054).

Similarly, any additional compounds or factors that are useful in the present invention include their analogs and variants that share a substantial similarity and at least one biological activity with the native compounds or factors. For example, EGF can be used in conjunction with growth hormone/IGF-1 in the present invention. In addition to native EGF, an EGF analog or variant can also be used, which should share a substantial amino acid sequence similarity with the native EGF, as well as at least one biological activity with the native EGF, such as binding to the EGF receptor. Particularly included as an EGF is the native EGF of any species, TGFα, or recombinant modified EGF. Specific examples include, but are not limited to, the recombinant modified EGF having a deletion of the two C-terminal amino acids and a neutral amino acid substitution at position 51 (particularly EGF51gln51; U.S. Patent Application Publication No. 20020098178A1), the EGF mutein (EGF-X₁₆) in which the His residue at position 16 is replaced with a neutral or acidic amino acid (U.S. Pat. No. 6,191,106), the 52-amino acid deletion mutant of EGF which lacks the amino terminal residue of the native EGF (EGF-D), the EGF deletion mutant in which the N-terminal residue as well as the two C-terminal residues (Arg-Leu) are deleted (EGF-B), the EGF-D in which the Met residue at position 21 is oxidized (EGF-C), the EGF-B in which the Met residue at position 21 is oxidized (EGF-A), heparin-binding EGF-like growth factor (HB-EGF), betacellulin, amphiregulin, neuregulin, or a fusion protein comprising any of the above. Other useful EGF analogs or variants are described in U.S. Patent Application Publication No. 20020098178A1, and U.S. Pat. Nos. 6,191,106 and 5,547,935.

As another example, PACAP can also be used as an additional factor in the present invention. Useful PACAP analogs and variants include, without being limited to, the 38 amino acid and the 27 amino acid variants of PACAP (PACAP38 and PACAP27, respectively), and the analogs and variants disclosed in, e.g., U.S. Pat. Nos. 5,128,242; 5,198,542; 5,208,320; 5,326,860; 5,623,050; 5,801,147 and 6,242,563.

Erythropoietin analogs and variants are disclosed, for example, in U.S. Pat. Nos. 6,048,971 and 5,614,184.

Further contemplated in the present invention are functional agonists of growth hormone, IGF-1, or additional factors useful in the present invention. These functional agonists bind to and activate the receptor of the native factor, although they do not necessarily share a substantial sequence similarity with the native factor. For example, maxadilan is a polypeptide that acts as a specific agonist of the PACAP type-1 receptor (Moro et al., 1997).

Functional agonists of EPO have been extensively studied. EMP1 (EPO mimetic peptide 1) is one of the EPO mimetics described in Johnson et al., 2000. Short peptide mimetics of EPO are described in, e.g., Wrighton et al., 1996 and U.S. Pat. No. 5,773,569. Small molecular EPO mimetics are disclosed in, e.g., Kaushansky, 2001. Antibodies that activate the EPO receptor are described in, e.g., U.S. Pat. No. 5,885,574; WO 96/40231 and WO 97/48729).

Antibodies that have agonist activities for the EGF receptor are described, e.g., in Fernandez-Pol, 1985 and U.S. Pat. No. 5,723,115. In addition, activating amino acid sequences are also disclosed in U.S. Pat. No. 6,333,031 for the EPO receptor, EGF receptor, prolactin receptor and many other cell surface receptors; metal complexed receptor ligands with agonist activities for the prolactin and EPO receptors can be found in U.S. Pat. No. 6,413,952. Other methods of identifying and preparing ligands for receptors, e.g., EPO and prolactin receptors, are described, for example, in U.S. Pat. Nos. 5,506,107 and 5,837,460.

It should be noted that the effective amount of each analog, variant or functional agonist may be different from that for the native factor or compound, and the effective amount in each case can be determined by a person of ordinary skill in the art according to the disclosure herein. Preferably, the native factors, or analogs and variants that share substantial sequence similarity with the native factors, are used in the present invention.

Pharmaceutical compositions are also provided, comprising a growth hormone and/or IGF-1, an additional factor as described above, and a pharmaceutically acceptable excipient and/or carrier.

The pharmaceutical compositions can be delivered via any route known in the art, such as parenterally, intrathecally, intravascularly, intravenously, intramuscularly, transdermally, intradermally, subcutaneously, intranasally, topically, orally, rectally, vaginally, pulmonarily or intraperitoneally. Preferably, the composition is delivered into the central nervous system by injection or infusion. More preferably it is delivered into a ventricle of the brain, particularly the lateral ventricle. Alternatively, the composition is preferably delivered by systemic routes, such as subcutaneous administrations. For example, we have discovered that prolactin, growth hormone, IGF-1, PACAP and EPO can be effectively delivered by subcutaneous administration to modulate the number of neural stem cells in the subventricular zone.

When the composition is not directly delivered into the brain, and factors in the composition do not readily cross the blood brain barrier, a blood brain barrier permeabilizer can be optionally included to facilitate entry into the brain. Blood brain barrier permeabilizers are known in the art and include, by way of example, bradykinin and the bradykinin agonists described in U.S. Pat. Nos. 5,686,416; 5,506,206 and 5,268,164 (such as NH₂-arginine-proline-hydroxyproxyproline-glycine-thienylalanine-serine-proline-4-Me-tyrosineψ(CH₂NH)-arginine-COOH). Alternatively, the factors can be conjugated to the transferrin receptor antibodies as described in U.S. Pat. Nos. 6,329,508; 6,015,555; 5,833,988 or 5,527,527. The factors can also be delivered as a fusion protein comprising the factor and a ligand that is reactive with a brain capillary endothelial cell receptor, such as the transferrin receptor (see, e.g., U.S. Pat. No. 5,977,307).

The pharmaceutical compositions can be prepared by mixing the desired therapeutic agents with an appropriate vehicle suitable for the intended route of administration. In making the pharmaceutical compositions of this invention, the therapeutic agents are usually mixed with an excipient, diluted by an excipient or enclosed within such a carrier which can be in the form of a capsule, sachet, paper or other container. When the pharmaceutically acceptable excipient serves as a diluent, it can be a solid, semi-solid, or liquid material, which acts as a vehicle, carrier or medium for the therapeutic agent. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the therapeutic agents, soft and hard gelatin capsules, suppositories, sterile injectable solutions, and sterile packaged powders.

Some examples of suitable excipients include artificial cerebral spinal fluid, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the therapeutic agents after administration to the patient by employing procedures known in the art.

For preparing solid compositions such as tablets, the therapeutic agent is mixed with a pharmaceutical excipient to form a solid preformulation composition containing a homogeneous mixture of a compound of the present invention. When referring to these preformulation compositions as homogeneous, it is meant that the therapeutic agents are dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein. The compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

Another formulation employed in the methods of the present invention employs transdermal delivery devices (“patches”). Such transdermal patches may be used to provide continuous or discontinuous infusion of the therapeutic agent of the present invention in controlled amounts. The construction and use of transdermal patches for the delivery of pharmaceutical agents is well known in the art. See, for example, U.S. Pat. 5,023,252, herein incorporated by reference. Such patches may be constructed for continuous, pulsatile, or on demand delivery of pharmaceutical agents.

Other suitable formulations for use in the present invention can be found in Remington's Pharmaceutical Sciences.

The following examples are offered to illustrate this invention and are not to be construed in any way as limiting the scope of the present invention.

### EXAMPLES

In the examples below, the following abbreviations have the following meanings. Abbreviations not defined have their generally accepted meanings.

| | |
|---|---|
| ° C. | degree Celsius |
| hr | hour |
| min | minute |
| μM | micromolar |
| mM | millimolar |
| M | molar |
| ml | milliliter |
| μl | microliter |
| mg | milligram |
| μg | microgram |
| kD | kilodalton |
| FBS | fetal bovine serum |
| PBS | phosphate buffered saline |
| DMEM | Dulbecco's modified Eagle's medium |
| α-MEM | α-modified Eagle's medium |
| β-ME | β-mercaptoethanol |
| EGF | epidermal growth factor |
| PDGF | platelet derived growth factor |
| GH | growth hormone |
| IGF-1 | insulin-like growth factor 1 |
| NSC | neural stem cell |
| SVZ | subventricular zone |
| RMS | rostral migratory stream |
| PACAP | pituitary adenylate cyclase activating polypeptide |
| cAMP | cyclic AMP |
| BMP | bone morphogenetic protein |
| OB | olfactory bulb |
| aCSF | artificial cerebral spinal fluid |

### Materials and Methods

Neural Stem Cell Culture

The protocols for neural stem cell culture are described in detail in U.S. Pat. No. 5,750,376 or Shingo et al., 2001. Briefly, embryonic neural stem cells were prepared from E14 medial and lateral ganglionic eminences. Adult neural stem cells were prepared from the subventricular zone of adult mice. The tissue was cultured in basal medium containing 20 ng/ml EGF, or other growth factors as indicated in each case, to form neurospheres. The composition of the basal medium is as follows: DMEM/F12 (1:1); glucose (0.6%); glutamine (2 mM); sodium bicarbonate (3 mM); HEPES (5 mM); insulin (25 μg/ml); transferrin (100 μg/ml); progesterone (20 nM); putrescine (60 μM); and selenium chloride (30 nM).

Seven days later, the neurospheres (primary neurospheres) were passaged by mechanical dissociation and reseeded as single cells (passage 1). For secondary neurospheres, the single cells were then cultured for seven days to form secondary neurospheres.

Test Animals for the Stroke Study

Adult male Long-Evans rats (250-350 g) were obtained from Charles River Breeding Farms (Laval, Quebec, Canada) and were adapted to the colony for two weeks prior to any treatment. A week before surgery the rats were given a baseline testing on the forelimb asymmetry test

Focal Ischemic Injury and Infusion

The animals for the stroke study received unilateral devascularization of the sensorimotor cortex. Using Isoflurane anesthesia, the skin was incised and retracted and the overlying fascia were removed from the skull. A skull opening was made at the following coordinates, taking care not to damage the dura: AP +4.0 to −2.0; L 1.5 to 4 (the parasagittal ridge; Kolb et al., 1997). The dura was cut and retracted from the skull opening. A cotton swab soaked in sterile saline was gently rubbed across the exposed pia and the vessels were removed. A hole was then drilled in the contralateral hemisphere to provide an opening for the cannulae attached to the osmotic minipump at AP−0.5; L 1.5. An osmotic minipump was placed under the skin between the shoulder blades and a tube connected under the skin to the cannulae, which was attached to the skull with fast-drying cement. Once hemostasis had been achieved the scalp was sutured closed with 5-O sterile suture. The animals were given a single injection of Banamine (an analgesic) and returned to their home cage. Sham animals received only anesthesia, the bone opening, and the skin was incised and sutured.

Six days later the animals were assessed using the behavioral test and on the following day the animals were re-anesthetized and the minipump was replaced with a second one containing the appropriate solutions. Sham animals were only anesthetized. The animals were retested 7, 14, and 28 days later to yield behavioral measures on weeks 1,2,3,4, and 6.

Forelimb Asymmetry Test

Forepaw asymmetry of the animals was determined by filming them from below while in an acrylic cylinder 25 cm in diameter, on an acrylic platform. Preference was determined by separately counting the number of times in 5 minutes that an animal reared and placed the left or right forepaw on the surface of the cylinder in a gesture of postural stabilization. This test allows a measure of asymmetry in forelimb use, a measure that shows a reliable bias to using the limb ipsilateral to the injury.

Brain Anatomical Analysis

At the conclusion of week 6 the animals were given an overdose of Euthanol and intracardially perfused with 0.9% saline and 4% paraformaldehyde in picric acid. The brains were cryoprotected and cut on a Vibratome at 40 microns. Five sets of sections were kept every 400 microns. Two sets were stained, one with Cresyl Violet and one with Doublecortin. The remaining sets were saved. The Cresyl Violet staining was performed on the slides whereas the Doublecortin was performed as an immunohistochemical procedure on free-floating sections. The Cresyl Violet staining allows assessment of lesion extent whereas the Doublecortin stains for a microtubule associated protein that is present in migrating neuronal progenitor cells.

### Example 1

Neural Stem Cell Number Declines Significantly in Aged Mice

To determine if the number of neural stem cells is affected by aging, the entire subventricular zones of the forebrain (both hemispheres) were collected from male and female C57BL/6J mice at various ages. The brain tissued were dissected, enzymatically dissociated and plated in defined culture medium in the presence of epidermal growth factor as described herein and in U.S. Pat. No. 5,750,376, and allowed to develop into primary neurospheres. Seven to ten days later, the numbers of neurospheres, each of which is clonally derived from a single stem cell, were counted.

The results (FIG. 1A) demonstrate that NSC numbers were reduced by 50-75% in the forebrain of aged mice (22-24 months old) in comparison to their young adult counterparts (2-4 months old). Male and female mice showed comparable reductions, indicating that the difference in sexual hormones is not the basis of this reduction.

Three other strains of mice were used to repeat this experiment to determine if this age-related reduction is a general phenomenon. As shown in FIG. 1B, CBA, DBA and Balb/c mice yielded similar patterns of NSC decline, indicating that NSC number reductions is commonly associated with aging.

The remaining question is whether the neural stem cell of aged animals have the same ability to self-renew and to differentiate into all lineages of neural cells. Therefore, the cells in the primary neurospheres were dissociated and allowed to generate secondary neurospheres, which is an indication of the ability to self-renew. The ability of the cells to differentiate into neurons, astrocytes and oligodendrocytes was also assessed by staining for specific markers of each cell type. The results (FIG. 3A) show that NSCs from aged mice were multipotent and able to differentiate into all three cell types, but their ability to self-renew was not as high as NSCs from their young adult counterparts. This impaired ability to self-renew is consistent with the reduction of NSC numbers with aging.

### Example 2

Reduced Proliferation in vivo in Aged Mice

The reduction of NSC numbers in aged mice may be resulted from decreased proliferation of neural stem cells when the animals get older. Therefore, BrdU was infused into the brain of young adults (2 months) or aged mice (24 months), and the number of BrdU positive cells in the subventricular zone or the rostral migratory stream were determined with BrdU specific antibodies. The subventricular zone is the primary location of neural stem cells in adult mammals, and the progeny of neural stem cells, neuron precursor cells and glial precursor cells, move along the rostral migratory stream to replenish the neurons in olfactory bulbs. Therefore, the ability of cells in the subventricular zone and the rostral migratory stream to incorporate BrdU is a good indication of neural stem cell proliferative activities.

The results are summarized in Table 1. Numbers of BrdU positive cells in both the subventricular zone and the rostral migratory stream were significantly reduced in aged mice, which is consistent with our previous results that neural stem cells numbers decline at old age, and that the self-renewal activity of aged neural stem cells is impaired. The number of periglomerular interneurons in aged olfactory bulbs, however, was higher than that in young adults. These results may indicate that a feedback control mechanism existing between the number of OB neurons and the number of neural stem cells. Thus, when there is a large quantity of periglomerular interneurons, proliferation of neural stems in the subventricular zone, as well neurogenesis in the rostral migratory stream, is down-regulated.

TABLE 1
Age-related changes in proliferating cells
in the SVZ and RMS and in total number of
perilogmerular olfactory bulb neurons

| | | | Total TH-IR |
|---|---|---|---|
| Age | BrdU cells in SVZ | BrdU cells in RMS | neurons in OB |
| 2 months | 1633 ± 36 | 399 ± 6 | 1710 ± 153 |
| 24 months | 415 ± 15* | 84 ± 8** | 2455 ± 258* |

Data are the means ± SEM for four animals in each group.
*Significantly different than 2 months, p < 0.05.
**p < 0.01.

### Example 3

Growth Hormone Induces SVZ Proliferation in vivo

To investigate if growth hormone is capable of inducing proliferation in the subventricular zone, where neural stem cells are primarily located in adult mammals, BrdU was infused with aCSF alone (control) or growth hormone and aCSF. The extent of BrdU incorporation was then determined with antibodies specific for BrdU. The results indicate that growth hormone significantly increased proliferation in the subventricular zone. Moreover, growth hormone also induced the newly-generated cells to migrate into the striatum.

### Example 4

Growth Hormone Receptor is Expressed in Adult Neurospheres

If growth hormone acts directly on neural stem cells to induce proliferation, neural stem cells should have growth hormone receptors. It is also possible that growth hormone induces the formation of IGF-1, which in turn induces proliferation of neural stem cells through IGF-1 receptors. Therefore, the levels of growth hormone receptors and IGF-1 receptors were determined with RT-PCR using RNA harvested from neurospheres and appropriate primers. The results show that both growth hormone and IGF-1 receptors were expressed robustly in neurospheres.

### Example 5

The Effect of Growth Hormone in a Stroke Model

In order to determine the effect of growth hormone in animals that suffer a brain injury, focal ischemic injuries were introduced into the brains of rats as a model of stroke. As a result of the brain injury, the animals had lesions in the motor cortex and behaved abnormally in the forelimb asymmetry test. Thus, while normal rats use both forelimbs equally when they try to balance themselves, these ischemic rats showed an asymmetry of paw use and preferred to use the ipsilateral paw, an expected result from the injury since the motor cortex controls the contralateral part of the body.

The animals then received various test factors, and the effects of these factors on the forelimb asymmetry test and brain anatomy were assessed. As controls, a sham control group received a sham brain injury and no test factors, and a vehicle control group received the brain injury as well as infusions of artificial cerebral spinal fluid (CSF). The treatments each test group received are summarized below:

| | | First Infusion | Second Infusion |
|---|---|---|---|
| Group | Brain Injury | (days 1-7) | (days 8-14) |
| 1 | sham | none | none |
| 2 | yes | CSF | CSF |
| 3 | yes | growth hormone | CSF |
| 4 | yes | growth hormone | erythropoietin (EPO) |

The schedule and procedure of the brain injury, infusion, behavioral test and anatomical analysis are described in Materials and Methods.

The results of the behavioral test indicate that although the extent of asymmetry decreased at the end of week six in all the test groups, the groups receiving growth hormone (Groups 3 and 4) showed a faster and more extensive recovery in the first four weeks. These results are consistent with those from the anatomical analysis, which show that growth hormone alone (Group 3) resulted in increased doublecortin positive cells, and the combination of growth hormone and EPO (Growth 4) led to migration of doublecortin positive cells around the lateral ventricle.

Accordingly, growth hormone, either alone or in conjunction with EPO, improved a motor neuron-related function in a stroke model as well as neuron formation/migration in the brain, indicating that growth hormone can be used to treat or ameliorate brain injuries.

We claim:

1. A method of increasing neural stem cell number, comprising providing an effective amount of a factor to at least one neural stem cell under conditions which result in an increase in the number of neural stem cells, wherein the factor is a growth hormone and/or insulin-like growth factor.

2. The method of claim 1 further comprising providing at least one additional factor to the neural stem cell.

3. The method of claim 2 wherein the additional factor is selected from the group consisting of erythropoietin, cyclic AMP, pituitary adenylate cyclase activating polypeptide (PACAP), serotonin, bone morphogenetic protein (BMP), epidermal growth factor (EGF), transforming growth factor alpha (TGFα), fibroblast growth factor (FGF), estrogen, prolactin, and ciliary neurotrophic factor (CNTF).

4. The method of claim 1 wherein the neural stem cell is cultured in vitro.

5. The method of claim 1 wherein the neural stem cell is located in the brain of a mammal.

6. The method of claim 5 wherein the neural stem cell is located in the subventricular zone of the brain.

7. The method of claim 6 wherein the factor is administered to the ventricle of the brain.

8. The method of claim 5 wherein the mammal is an adult mammal.

9. The method of claim 5 wherein the mammal suffers from or is suspected of having a neurodegenerative disease or condition.

10. The method of claim 9 wherein the disease or condition is a brain injury.

11. The method of claim 10 wherein the brain injury is a stroke.

12. The method of claim 10 wherein the brain injury is associated with brain surgery.

13. The method of claim 9 wherein the neurodegenerative disease or condition is selected from the group consisting of Alzheimer's disease, multiple sclerosis, Huntington's disease, amyotrophic lateral sclerosis, and Parkinson's disease.

14. The method of claim 9 wherein the mammal receives a transplantation of neural stem cells and/or neural stem cell progeny prior to or concurrently with the factor.

15. The method of claim 9 wherein the factor is provided to the mammal by administering the factor intravascularly, intrathecally, intravenously, intramuscularly, subcutaneously, intraperitoneally, topically, orally, rectally, vaginally, nasally, by inhalation or into the brain.

16. The method of claim 9 wherein the factor is administered subcutaneously.
